(19) United States
(12) Patent Application Publication
(10) Pub. No.: US 2009/0312731 A1
(43) Pub. Date: Dec. 17, 2009

(54) Nonwoven Melt-Blown Product

(75) Inventors: Thomas Steindl, Neusiedl am See (AT); Mirko Einzmann, Wels (AT); Shayda Rahbaran, Vocklabruck (AT); Josef Schmidtbauer, Vocklabruck (AT); Bernhard Voglauer, Timelkam (AT)

Correspondence Address: FITZPATRICK CELLA HARPER & SCINTO, 1290 Avenue of the Americas, NEW YORK, NY, 10104-3800, US

(73) Assignee: LENZING AKTIENGESELLSCHAFT, Lenzing (AT)

(21) Appl. No.: 12/298,084

(22) PCT Filed: Apr. 26, 2007
(86) PCT No.: PCT/AT2007/000193

§ 371 Date: Feb. 13, 2009

(30) Foreign Application Priority Data
Apr. 28, 2006 (AT) A727/2006
Apr. 28, 2006 (AT) A729/2006
Apr. 28, 2006 (AT) A730/2006
Apr. 28, 2006 (AT) A731/2006

(51) Int. Cl. A61F 13/53; D04H 13/00; B32B 5/00; B29C 47/00; D04H 3/08; B01D 39/16

(52) U.S. Cl. 604/374; 442/400; 428/220; 428/219; 264/176.1; 156/180; 55/528; 977/773

(57) ABSTRACT

The present invention relates to a product selected from the group consisting of filter products, disposable absorbent products, wipe products and structural and/or reinforcement products characterized in that the product is made of or includes a nonwoven web made by a melt-blowing process employing a cellulose solution in N-methyl-morpholine (NMMO).

### CROSS-REFERENCE TO RELATED APPLICATIONS

This application claims priority to International Patent Application No. PCT/AT2007/000193, filed on Apr. 26, 2007, which claims priority to Austrian Patent Application Nos. A727/2006, A729/2006, A730/2006 and A731/2006, all filed on Apr. 28, 2006.

### FIELD OF THE INVENTION

The present invention relates to a product selected from the group consisting of filter products, disposable absorbent products, wipe products and structural and/or reinforcement products.

### BACKGROUND OF THE INVENTION

It is known to produce nonwoven webs made by a melt-blowing process employing a cellulose solution in N-methyl-morpholine-N-oxide (“NMMO”) from, inter alia, WO 98/26122, WO 99/47733, WO 98/07911, U.S. Pat. No. 6,197,230, WO 99/64649, WO 05/106085 and EP 1 358 369.

Under a “melt-blowing process” one understands a process, wherein extruded threads of the cellulose solution in NMMO are picked up by a gas stream flowing in a generally parallel direction to the path of the filaments. The cellulose solution, which is ejected through the orifices, is formed to liquid strands or latent filaments, which are drawn (or significantly decreased in diameter and increased in length and may be split into several sub-filaments) by the gas stream.

The filaments are then collected, for example on a rotating drum, whereby a web is formed.

In the following, this process is referred to as “Lyocell melt-blowing process”. Nonwoven melt-blown webs made by the Lyocell melt-blowing process will in the following be referred to as “Lyocell melt-blown webs”. For the purposes of the present invention, the term “Lyocell melt-blowing process” encompasses both processes by which endless filaments are obtained (such processes also referred to in the literature as “spunbonding processes”), processes by which fibres of a discrete length are obtained and processes by which mixtures of endless filaments and fibres of discrete length are obtained.

### SUMMARY OF THE INVENTION

It has now been found that Lyocell melt-blown webs are very useful in a product selected from the group consisting of filter products, disposable absorbent products, wipe products and structural and/or reinforcement products.

Therefore, the present invention relates to a product selected from the group consisting of filter products, disposable absorbent products, wipe products and structural and/or reinforcement products. which is characterized in that it is made of or comprises a nonwoven web made by a melt-blowing process employing a cellulose solution in N-methyl-morpholine (NMMO).

Preferred embodiments are disclosed in the dependent claims.

### DETAILED DESCRIPTION OF THE INVENTION

The Lyocell melt-blown web employed in the filter products according to the present invention preferably exhibits one or more of the following properties:

(i) the dry weight of the web according to WSP 130.1 (05) is from 30 to 500 g/m², preferably 40 to 80 g/m²

(ii) the tenacity of the web according to WSP 110.4 (05) is from 5 to 300 N, preferably 10 to 120 N

(iii) the elongation of the web according to WSP 110.4 (05) is from 5% to 300%

(iv) the thickness of the web is from 0.2 to 0.7 mm, preferably 0.3 to 0.7 mm

(v) the density of the web is from 0.07 to 0.80 g/cm³, preferably from 0.07 to 0.20 g/cm³

Most preferably, the Lyocell melt-blown web exhibits all of said properties (i) to (v) mentioned above.

The Lyocell melt-blown web employed in the disposable absorbent products according to the present invention preferably exhibits one or more of the following properties:

If used for a tampon:

(i) The syngina value according to WSP 350.1 (05) is from 3 to 27 g/tampon, preferably from 6 to 24 g/tampon

(ii) The dry weight of the web according to WSP 130.1 (05) is between 120 g/m² and 220 g/m², preferably 150 g/m² to 200 g/m²

If used for other absorbent products:

(iii) The dry weight of the web according to WSP 130.1 (05) is preferably from 9 to 300 g/m², preferably 9 to 200 g/m²

(iv) The water retention value of the web according to DIN 53814 is from 1 to 300%, preferably 30 to 100%

(v) The liquid absorbency capacity of the web according to WSP 10.1 (05) is from 2 to 2000%, preferably 250 to 1500%.

Most preferably, the Lyocell melt-blown web exhibits all of said properties (i) to (ii), or (iii) to (v), respectively, as mentioned above.

The Lyocell melt-blown web employed in the wipe products according to the present invention preferably exhibits one or more of the following properties:

(i) the dry weight of the web is from 10 to 300 g/m², preferably 40-80 g/m²

(ii) the thickness of the web according to WSP 120.6 (05) is from 0.2 to 5.0 mm, preferably 0.3 to 0.7 mm

(iii) the tenacity of the web according to WSP 110.4 (05) is from 5 to 250 N, preferably 10 to 120 N

(iv) the tenacity of the web in Nm²/g is from 0.1 to 3.0, preferably from 0.7 to 2.3

(v) the elongation of the web according to WSP 110.4 (05) is from 5 to 300%

(vi) the MD/CD tenacity ratio of the web is from 1 to 12

(vii) The water retention of the web according to DIN 53814 is from 1 to 250%, preferably 30 to 150%

(viii) The liquid absorbency capacity of the web according to WSP 10.1 (05) is 90 to 2000%, preferably 400 to 1000%.

Most preferably, the Lyocell melt-blown web exhibits all of said properties (i) to (viii) mentioned above.

The Lyocell melt-blowing process for producing the webs used in the present invention preferably comprises the steps of

(a) extruding a solution comprising cellulose dissolved in NMMO through a spinneret into an air gap, thereby forming filaments

(b) drawing said filaments by means of a gaseous stream

(c) collecting and precipitating said filaments in order to form a web

(d) converting said web into a filter product.

These steps are generally known as such from the documents mentioned above, to which reference is made in this regard. The skilled artisan is well aware of the various possibilities to carry out this process in order to achieve useful Lyocell melt-blown webs.

Especially in the case of filter products, disposable absorbent products and structural and/or reinforcement products according to the invention, the Lyocell melt-blowing process preferably comprises the further step of

(e) precipitating said filaments only after having been formed into a web.

By way of this step (e), the filaments are only precipitated after having been collected such as to form a web. It has been shown that this measure imparts to the webs thusly produced a more “paper-like” performance and high tenacities, as compared to the more soft and textile-like performance of products where the filaments are at least partly precipitated before after forming the web.

In the case of disposable absorbent products, webs produced according to this embodiment may for example be used as an inlay in absorbent products, such as diapers.

In another alternative, and especially in the case of disposable absorbent products and wipe products according to the invention, the Lyocell melt-blowing process preferably comprises the further step of

(e) contacting said filaments with a vapour mist before collecting said filaments, thereby at least partly precipitating said filaments.

As mentioned above, webs produced according to this alternative have a more soft and textile-like performance, therefore, e.g. in the case of disposable absorbent products, rendering them suitable for products such as tampons or as a skin-contacting layer in other absorbent products.

The properties of the Lyocell melt-blown web can be further enhanced if the production process comprises the further step of

(f) bonding said web by means of a hydroentanglement process before converting said web into said filter product.

It has been found that it is well possible to bond a Lyocell melt-blown web by way of a hydroentanglement process.

Lyocell melt-blown webs produced with the preferred Lyocell melt-blowing process comprising both the steps (e) and (f) as mentioned above are especially useful as a filter product, a disposable absorbent product, a structural and/or reinforcement product or, at least, a part of said filter, disposable absorbent, structural and/or reinforcement product product.

Lyocell melt-blown webs produced with the preferred Lyocell melt-blowing process comprising both the steps (e′), and (f), as mentioned above, are especially useful as a disposable absorbent product or a wipe product or, at least, a part of said disposable absorbent or wipe product.

The Lyocell melt-blown webs, alternatively may be bonded by other means of bonding, such as chemical bonding, thermal bonding and needle-punching.

In order to be processed to a filter product, the Lyocell melt-blown webs may be combined with layers of other materials, such as:

In the case of filter products: Layers of synthetic polymers, cellulosic fluff pulp, nonwoven webs of cellulose or synthetic polymer fibers, bicomponent fibres, webs of cellulose pulp, such as airlaid pulp, webs or fabrics of high tenacity fibres, hydrophobic materials, high performance fibres (such as temperature resistant materials or flame retardant materials, layers imparting changed mechanical properties to the final products (such as Polypropylene or Polyester layers), biodegradable materials (e.g. films, fibres or webs from Polylactic acid), and/or high bulk materials (such as Polyacrylnitrile).

In the case of disposable absorbent products: Layers of synthetic polymers, cellulosic fluff pulp, nonwoven webs of cellulose or synthetic polymer fibers, and webs of cellulose pulp, such as airlaid pulp.

In the case of wipe products: Layers of synthetic polymers, cellulosic fluff pulp, nonwoven webs of cellulose or synthetic polymer fibers, bicomponent fibres, webs of cellulose pulp, such as airlaid pulp, webs or fabrics of high tenacity fibres, hydrophobic materials, high performance fibres (such as temperature resistant materials or flame retardant materials, layers imparting changed mechanical properties to the final products (such as Polypropylene or Polyester layers), biodegradable materials (e.g. films, fibres or webs from Polylactic acid), and/or high bulk materials (such as Polyacrylnitrile).

In the case of structural and/or reinforcement products: Layers of synthetic polymers, cellulosic fluff pulp, nonwoven webs of cellulose or synthetic polymer fibers, bicomponent fibres, webs of cellulose pulp, such as airlaid pulp, webs or fabrics of high tenacity fibres, hydrophobic materials, high performance fibres (such as bullet proof materials, temperature resistant materials or flame retardant materials, layers imparting changed mechanical properties to the final products (such as Polypropylene or Polyester layers), biodegradable materials (e.g. films, fibres or webs from Polylactic acid), and/or high bulk materials (such as Polyacrylnitrile).

It is also possible to combine several layers of Lyocell melt-blown webs.

In a further preferred embodiment of the present invention, the Lyocell melt-blown web contained in the products according to the invention essentially consists of cellulose.

In an alternative embodiment, the Lyocell melt-blown web comprises a mixture of cellulose and one or more other fiber materials. WO 98/07911, for example, proposes the use of mixtures of cellulose and other materials, such as non-cellulosic polymers, for the production of melt-blown products.

The Lyocell melt-blown web, furthermore, may comprise a bicomponent fiber material.

The fiber material in the Lyocell melt-blown web may at least partly comprise a modifying substance.

In the case of filter products, the modifying substance may be selected from the group consisting of antibacterial products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, dye stuffs, and resins for improvement of the chemical and mechanical stability.

In the case of disposable absorbent products, the modifying substance may be selected from the group consisting of antibacterial products, ion-exchanger products, active carbon, nanoparticles, lotions, fire-retardant products and dye-stuffs.

In the case of wipe products, the modifying substance may be selected from the group consisting of antibacterial products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, and dye stuffs.

In the case of structural and/or reinforcement products, the modifying substance may be selected from the group consisting of antibacterial products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, dye stuffs, and resins for improvement of the chemical and mechanical stability.

The filter product according to the present invention preferably is a product selected from the group consisting of air filters, e.g. HVAC, HEPA and ULPA filters, flue gas filters, liquid filters, coffee filters, tea bags, coffee bags, food filters, water purification filters, blood filters, cigarette filters, cabin filters, fuel filters, oil filters, cartridge filters, vacuum filters, vacuum cleaner bags, dust filters, hydraulic filters, kitchen filters, fan filters, beer filters, milk filters, liquid coolant filters, fruit juices filters, face masks, and active carbon filters.

The disposable absorbent product according to the present invention preferably is a product selected from the group consisting of acquisition layers, coverstock, distribution layers, absorbent covers, sanitary napkins, panty liners, diapers, incontinence products, towels, tampons, sanitary pads, backsheets, leg cuffs, flushable products, pads, nursing pads, disposal underwear, training pants, cosmetic removal pads and washcloths.

As regards tampons, the Lyocell melt-blown web may both used in digital and in application tampons.

A Lyocell melt-blown web with a weight between 120 g/m² and 220 g/m², preferably 150 g/m² to 200 g/m² may be processed in a tampon directly after having been produced, i.e. without intermediate steps.

The density of tampons produced from the Lyocell melt-blown web may range from 0.1 to 0.8 g/cm³, preferably 0.3 to 0.6 g/cm³.

Furthermore, a very thin Lyocell melt-blown web with a weight of only about 8 g/m² may be used as a wrapping material for tampons.

The wipe product according to the present invention preferably is a product selected from the group consisting of baby wipes, kitchen wipes, wet wipes, cosmetic wipes, hygiene wipes, cleaning wipes, glass wipes, lens cleaning wipes, polishing wipes, e.g. for cars and furniture, dust wipes, industrial wipes, intimate wipes, toilet wipes, floor wipes, glass wipes, medical wipes, such as cleansing and pre-operative wipes, dusters and mops.

Reinforcing materials comprising the Lyocell melt-blown web may be produced by dipping the Lyocell melt-blown web into a polymer mass, or by sucking a polymer into a web, whereby the cellulose fibers in the composite material thereby obtained act as reinforcing material.

The Lyocell melt-blown web, or products made therefrom, may be carbonized in order to form a carbonized fibre web.

The structural and/or reinforcement product according to the present invention preferably is a product selected from the group consisting of geotextile products, such as capillary matting, water purification products, irrigation control products, asphalt overlay, soil stabilisation products, drainage products, e.g. drainage channel liners, sedimentation and erosion control products, pond liners, impregnation base products, ground stabilisation products, pit linings, seed blankets, weed control fabrics, greenhouse shading, root bags and biodegradable plant pots; building products, such as roofing and tile underlay, understating, thermal and noise insulation, house wraps, facings for plaster board, pipe wraps, concrete moulding layers, foundations and ground stabilisation products, vertical drainages, shingles, roofing felts, noise abatement materials, reinforcement materials, sealing materials, mechanical damping materials, fire protection materials, reinforced concrete and reinforced polymers; automotive products, such as cabin filters, boot liners, parcel shelves, heat shields, shelf trim, moulded bonnet liners, boot floor covering, oil filters, headliners, rear parcel shelves, decorative fabrics, airbags, silencer pads, insulation materials, car covers, underpadding, car mats, tapes, backing and tufted carpets, seat covers, door trim, needled carpets, auto carpet backings, and reinforced polymers; furnishing products, such as furniture constructions, insulators to arms and backs, cushion thickings, dust covers, linings, stitch reinforcements, edge trim materials, bedding constructions, quilt backing, spring wraps, mattress pad components, mattress covers, window curtains, wall coverings, carpet backings, lampshades, mattress components, spring insulators, sealings, pillow thicking, mattress thicking and high loft webs for filling applications such as disposable duvets; industrial products, such as electronic products, floppy disc liners, cable insulation, abrasives, insulation tapes, conveyor belts, noise absorbent layers, air conditioning products, battery separators, acid systems, anti slip mattings, stain removers, food wraps, adhesive tapes, sausage casings, cheese casing, artificial leather, oil recovery booms and socks, papermaking felts, kettle descaler bags, and flame barriers; travel or leisure products, such as sleeping bags, tents, luggage, handbags, shopping bags, airline headrests, CD-protection products, pillowcases, sandwich packaging and surf boards; and school or office products, such as book covers, mailing envelopes, maps, signs and pennants, towels, flags and bank notes.

1. A product selected from the group consisting of filter products, disposable absorbent products, wipe products and structural and/or reinforcement products, wherein said product comprises a nonwoven web made by a melt-blowing process employing a cellulose solution in N-methyl-morpholine (NMMO).

2. The product according to claim 1, wherein said nonwoven web exhibits one or more of the following properties:
(i) the dry weight of the web according to WSP 130.1 (05) is from 30 to 500 g/m², preferably 40 to 80 g/m²
(ii) the tenacity of the web according to WSP 110.4 (05) is from 5 to 300 N, preferably 10 to 120 N.
(iii) the elongation of the web according to WSP 110.4 (05) is from 5% to 300%
(iv) the thickness of the web is from 0.2 to 0.7 mm, preferably 0.3 to 0.7 mm
(v) the density of the web is from 0.07 to 0.80 g/cm³, preferably from 0.07 to 0.20 g/cm³

3. The product according to claim 1 or 2, wherein said nonwoven web has been produced by a process comprising the steps of
(a) extruding a solution comprising cellulose dissolved in NMMO through a spinneret into an air gap, thereby forming filaments,
(b) drawing said filaments by means of a gaseous stream,
(c) collecting and precipitating said filaments to form a web, and,
(d) converting said web into a filter product.

4. The product according to claim 3, wherein said process further comprises the step of
(e) precipitating said filaments after said web is formed.

5. The product according to claim 3 or 4, wherein said process further comprises the step of
(f) bonding said web by means of a hydroentanglement process before converting said web into said filter product.

6. The product according to any of the preceding claims, wherein said nonwoven web consists essentially of cellulose.

7. The product according to any of claims 1 to 5, wherein said nonwoven web comprises a mixture of cellulose and one or more other fiber materials.

8. The product according to any of claims 1 to 5, wherein said nonwoven web comprises a bicomponent fiber material.

9. The product according to any of the preceding claims, wherein the fiber material in the nonwoven web at least partly comprises a modifying substance.

10. The product according to claim 9, wherein said modifying substance is selected from the group consisting of antibacterial products, ion-exchanger products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, dye stuffs, and resins for improvement of the chemical and mechanical stability.

11. The product according to any of the preceding claims, wherein said product is said filter product which is selected from the group consisting of air filters, such as HVAC, HEPA and ULPA filters, flue gas filters, liquid filters, coffee filters, tea bags, coffee bags, food filters, water purification filters, blood filters, cigarette filters, cabin filters, fuel filters, oil filters, cartridge filters, vacuum filters, vacuum cleaner bags, dust filters, hydraulic filters, kitchen filters, fan filters, beer filters, milk filters, liquid coolant filters, fruit juices filters, face masks, and active carbon filters.

12. The product according to claim 1, wherein said nonwoven web exhibits one or more of the following properties:
(A) If used for a tampon:
(i) The syngina value according to WSP 350.1 (05) is from 3 to 27 g/tampon, preferably from 6 to 24 g/tampon
(ii) The dry weight of the web according to WSP 130.1 (05) is between 120 g/m² and 220 g/m², preferably 150 g/m² to 200 g/m²
(B) If used for other absorbent products:
(iii) The dry weight of the web according to WSP 130.1 (05) is preferably from 9 to 300 g/m², preferably 9 to 200 g/m²
(iv) The water retention value of the web according to DIN 53814 is from 1 to 300%, preferably 30 to 100%
(v) The liquid absorbency capacity of the web according to WSP 10.1 (05) is from 2 to 2000%, preferably 250 to 1500%

13. The product according to claim 1, wherein said nonwoven web has been produced by a process comprising the steps of
(a) extruding a solution comprising cellulose dissolved in NMMO through a spinneret into an air gap, thereby forming filaments
(b) drawing said filaments by means of a gaseous stream
(c) collecting and precipitating said filaments in order to form a web
(d) converting said web into a disposable absorbent product.

14. The product according to claim 13, wherein said process further comprises the step of
(e) precipitating said filaments after said web is formed.

15. The product according to claim 13, wherein said process further comprises the step of
(e′) contacting said filaments with a vapour mist before collecting said filaments, thereby at least partly precipitating said filaments.

16. The product according to any of claims 13 to 15, wherein said process further comprises the step of
(f) bonding said web by means of a hydroentanglement process before converting said web into said disposable absorbent product.

17. The product according to any of claims 12 to 16, wherein said nonwoven web essentially consists of cellulose.

18. The product according to any of claims 12 to 16, wherein said nonwoven web comprises a mixture of cellulose and one or more other fiber materials.

19. The product according to any of claims 12 to 16, wherein said nonwoven web comprises a bicomponent fiber material.

20. The product according to or any of claims 12 to 19, wherein the fiber material in the nonwoven web at least partly comprises a modifying substance.

21. The product according to claim 20, wherein said modifying substance is selected from the group consisting of antibacterial products, ion-exchanger products, active carbon, nanoparticles, lotions, fire-retardant products and dye-stuffs.

22. The product according to claim 1 or any of claims 12 to 21, wherein said product is an absorbent product selected from the group consisting of acquisition layers, coverstock, distribution layers, absorbent covers, sanitary napkins, panty liners, diapers, incontinence products, towels, tampons, sanitary pads, backsheets, leg cuffs, flushable products, pads, nursing pads, disposal underwear, training pants, cosmetic removal pads and washcloths.

23. The product according to claim 1, wherein said nonwoven web exhibits one or more of the following properties:
(i) the dry weight of the web is from 10 to 300 g/m², preferably 40-80 g/m²
(ii) the thickness of the web according to WSP 120.6 (05) is from 0.2 to 5.0 mm, preferably 0.3 to 0.7 mm
(iii) the tenacity of the web according to WSP 110.4 (05) is from 5 to 250 N, preferably 10 to 120 N
(iv) the tenacity of the web in Nm²/g is from 0.1 to 3.0, preferably from 0.7 to 2.3
(v) the elongation of the web according to WSP 110.4 (05) is from 5 to 300%
(vi) the MD/CD tenacity ratio of the web is from 1 to 12
(vii) the water retention of the web according to DIN 53814 is from 1-200%, preferably 30 to 150%
(viii) The liquid absorbency capacity of the web according to WSP 10.1 (05) is 90 to 2000%, preferably 400 to 1000%.

24. The product according to claim 1 or 23, wherein said nonwoven web has been produced by a process comprising the steps of
(a) extruding a solution comprising cellulose dissolved in NMMO through a spinneret into an air gap, thereby forming filaments
(b) drawing said filaments by means of a gaseous stream
(c) collecting and precipitating said filaments in order to form a web
(d) converting said web into a wipe product.

25. A product according to claim 24, wherein said process further comprises the step of
(e) contacting said filaments with a vapour mist before collecting said filaments, thereby at least partly precipitating said filaments.

26. The product according to claim 24 or 25, wherein said process further comprises the further step of
(f) bonding said web by means of a hydroentanglement process before converting said web into said wipe product.

27. The product according to any of claims 23 to 26, wherein said nonwoven web consists essentially of cellulose.

28. The product according to any of claims 23 to 26, wherein said nonwoven web comprises a mixture of cellulose and one or more other fiber materials.

29. The product according to any of claims 23 to 26, wherein said nonwoven web comprises a bicomponent fiber material.

30. The product according to any of claims 23 to 29, wherein the fiber material in the nonwoven web at least partly comprises a modifying substance.

31. The product according to claim 30, wherein said modifying substance is selected from the group consisting of antibacterial products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, dye stuffs.

32. The product according to claim 1 or any of claims 23 to 31, wherein said product is said wipe product selected from the group consisting of baby wipes, kitchen wipes, wet wipes, cosmetic wipes, hygiene wipes, cleaning wipes, glass wipes, lens cleaning wipes, polishing wipes, such as for cars and furniture, dust wipes, industrial wipes, intimate wipes, toilet wipes, floor wipes, glass wipes, medical wipes, such as cleansing and pre-operative wipes, dusters and mops.

33. The product according to claim 1, wherein said nonwoven web has been produced by a process comprising the steps of
(a) extruding a solution comprising cellulose dissolved in NMMO through a spinneret into an air gap, thereby forming filaments
(b) drawing said filaments by means of a gaseous stream
(c) collecting and precipitating said filaments in order to form a web
(d) converting said web into a structural and/or reinforcement product.

34. The product according to claim 33, wherein said process further comprises the step of
(e) precipitating said filaments after said web is formed.

35. The product according to claim 33 or 34, wherein said process further comprises the step of
(f) bonding said web by means of a hydroentanglement process before converting said web into said structural and/or reinforcement product.

36. The product according to any of claims 33 to 35, wherein said nonwoven web consists essentially of cellulose.

37. The product according to any of claims 33 to 35, wherein said nonwoven web comprises a mixture of cellulose and one or more other fiber materials.

38. The product according to any of claims 33 to 37, wherein said nonwoven web comprises a bicomponent fiber material.

39. The product according to any of claims 33 to 38, wherein the fiber material in the nonwoven web at least partly comprises a modifying substance.

40. The product according to claim 39, wherein said modifying substance is selected from the group consisting of antibacterial products, nanoparticles, lotions, fire-retardant products, absorbency-improving additives, such as superabsorbent resins, ion-exchange resins, carbon resins such as active carbon, graphite, carbon for electrical conductivity; X-ray contrast resins, luminescent pigments, dye stuffs, and resins for improvement of the chemical and mechanical stability.

41. The product according to claim 1 or any of claims 33 to 40, wherein said product is said structural and/or reinforcement selected from the group consisting of geotextile products, such as capillary matting, water purification products, irrigation control products, asphalt overlay, soil stabilisation products, drainage products, e.g. drainage channel liners, sedimentation and erosion control products, pond liners, impregnation base products, ground stabilisation products, pit linings, seed blankets, weed control fabrics, greenhouse shading, root bags and biodegradable plant pots; building products, such as roofing and tile underlay, understating, thermal and noise insulation, house wraps, facings for plaster board, pipe wraps, concrete moulding layers, foundations and ground stabilisation products, vertical drainages, shingles, roofing felts, noise abatement materials, reinforcement materials, sealing materials, mechanical damping materials, fire protection materials, reinforced concrete and reinforced polymers; automotive products, such as cabin filters, boot liners, parcel shelves, heat shields, shelf trim, moulded bonnet liners, boot floor covering, oil filters, headliners, rear parcel shelves, decorative fabrics, airbags, silencer pads, insulation materials, car covers, underpadding, car mats, tapes, backing and tufted carpets, seat covers, door trim, needled carpets, auto carpet backings, and reinforced polymers; furnishing products, such as furniture constructions, insulators to arms and backs, cushion thickings, dust covers, linings, stitch reinforcements, edge trim materials, bedding constructions, quilt backing, spring wraps, mattress pad components, mattress covers, window curtains, wall coverings, carpet backings, lampshades, mattress components, spring insulators, sealings, pillow thicking, mattress thicking and high loft webs for filling applications such as disposable duvets; industrial products, such as electronic products, floppy disc liners, cable insulation, abrasives, insulation tapes, conveyor belts, noise absorbent layers, air conditioning products, battery separators, acid systems, anti slip mattings, stain removers, food wraps, adhesive tapes, sausage casings, cheese casing, artificial leather, oil recovery booms and socks, papermaking felts, kettle descaler bags, and flame barriers; travel or leisure products, such as sleeping bags, tents, luggage, handbags, shopping bags, airline headrests, CD-protection products, pillowcases, sandwich packaging and surf boards; and school or office products, such as book covers, mailing envelopes, maps, signs and pennants, towels, flags and bank notes.
